(19) United States
(12) Patent Application Publication
(10) Pub. No.: US 2024/0290426 A1
(43) Pub. Date: Aug. 29, 2024

(54) GENETIC CHARACTERISTIC ESTIMATION DEVICE, CONTROL METHOD, AND NON-TRANSITORY COMPUTER-READABLE MEDIUM

(71) Applicants: NEC Corporation, Tokyo (JP); OSAKA UNIVERSITY, Suita-shi, Osaka (JP)

(72) Inventors: Masataka KIKUCHI, Osaka (JP); Kenichi KAMIJO, Tokyo (JP); Yasuto FUSHIMI, Tokyo (JP); Kaori KOBAYASHI, Tokyo (JP)

(73) Assignees: NEC Corporation, Tokyo (JP); OSAKA UNIVERSITY, Suita-shi, Osaka (JP)

(21) Appl. No.: 18/569,733

(22) PCT Filed: Jun. 14, 2021
(86) PCT No.: PCT/JP2021/022428

§ 371 Date: Dec. 13, 2023

(51) Int. Cl. G16B 20/50

(52) U.S. Cl. CPC G16B 20/50

(57) ABSTRACT

A genetic characteristic estimation device (2000) acquires genetic mutation information (30) and position information (40). The genetic mutation information (30) indicates information regarding a genetic mutation in a deoxyribonucleic acid (DNA) sequence of a target cell (20) obtained from a target organism (10). In the position information (40), a position on the DNA sequence is associated with the type of a cell or the type of an organ. The genetic characteristic estimation device (2000) determines a genetic mutation at the position associated with the type of the target cell (20) or the type of an organ including the target cell (20) in the position information (40) from among genetic mutations indicated by the genetic mutation information (30), and computes a genetic characteristic index value representing a genetic characteristic of the target organism (10) based on a characteristic of the determined genetic mutation.

### TECHNICAL FIELD

The present disclosure relates to a technique for estimating a genetic characteristic of an organism.

### BACKGROUND ART

Techniques for estimating a genetic characteristic of an organism have been developed. For example, Patent Literature 1 discloses a technique for predicting a trait of an evaluation target from a genetic mutation of the evaluation target by using a database that stores information regarding genetic mutations common in a sample group showing a common trait. The system of Patent Literature 1 uses the information in the database to compute, for each of one or more genetic mutations of the evaluation target, a score indicating the degree of relevance between the genetic mutation and a specific trait, and predicts the trait based on the score.

### CITATION LIST

### Patent Literature

Patent Literature 1: International Patent Publication No. WO 2019/181022

### SUMMARY OF INVENTION

### Technical Problem

An objective of the present disclosure is to provide a new technique for estimating a genetic characteristic of an organism.

### Solution to Problem

A genetic characteristic estimation device of the present disclosure includes: an acquisition unit configured to acquire genetic mutation information regarding a genetic mutation in a deoxyribonucleic acid (DNA) sequence of a target cell obtained from a target organism, and position information that associates a position in the DNA sequence with a type of a cell or a type of an organ; and a computation unit configured to determine a mutation of interest, which is a genetic mutation at a position that the position information associates with the type of the target cell or the type of the organ having the target cell, from the genetic mutations indicated by the genetic mutation information, and compute a genetic characteristic index value, which represents a genetic characteristic of the target organism, based on a characteristic of the mutation of interest.

A control method of the present disclosure is executed by a computer. The control method includes: an acquisition step of acquiring genetic mutation information regarding a genetic mutation in a deoxyribonucleic acid (DNA) sequence of a target cell obtained from a target organism, and position information that associates a position in the DNA sequence with a type of a cell or a type of an organ; and a computation step of determining a mutation of interest, which is a genetic mutation at a position that the position information associates with the type of the target cell or the type of the organ having the target cell, from the genetic mutations indicated by the genetic mutation information, and computing a genetic characteristic index value, which represents a genetic characteristic of the target organism, based on a characteristic of the mutation of interest.

A non-transitory computer-readable medium of the present disclosure stores a program for causing a computer to execute the control method of the present disclosure.

### Advantageous Effects of Invention

According to the present disclosure, a new technique for estimating a genetic characteristic of an organism is provided.

### BRIEF DESCRIPTION OF DRAWINGS

FIG. 1 is a diagram illustrating an overview of an operation of a genetic characteristic estimation device according to a first example embodiment.

FIG. 2 is a block diagram illustrating a functional configuration of the genetic characteristic estimation device of the first example embodiment.

FIG. 3 is a block diagram illustrating a hardware configuration of a computer that implements the genetic characteristic estimation device.

FIG. 4 is a flowchart illustrating a flow of processes executed by the genetic characteristic estimation device of the first example embodiment.

FIG. 5 is a diagram illustrating genetic mutation information in a table format.

FIG. 6 is a diagram illustrating position information in a table format.

FIG. 7 is a diagram illustrating contribution degree information in a table format.

### EXAMPLE EMBODIMENT

Hereinafter, example embodiments of the present disclosure will be described in detail with reference to the drawings. In the drawings, the same or corresponding elements are denoted by the same reference numerals and signs and redundant explanation of the elements is omitted according to necessity for clarity of explanation. Further, unless otherwise described, a predefined value, such as a predefined value or threshold, is stored in advance in a storage unit or the like in such a manner that a device using the value can acquire it. Furthermore, unless otherwise described, the storage unit includes one or more storage devices.

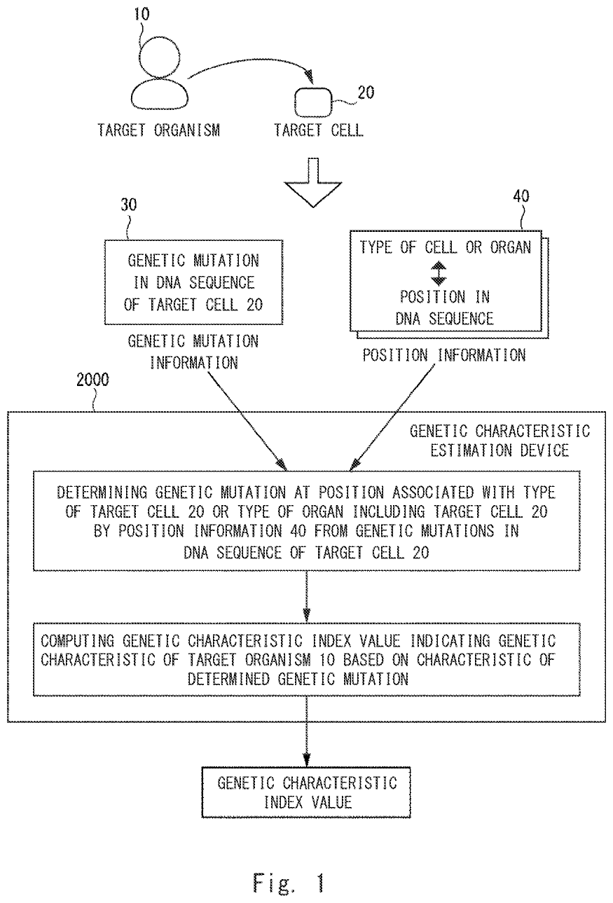

FIG. 1 is a diagram illustrating an overview of an operation of a genetic characteristic estimation device 2000 according to a first example embodiment. Here, FIG. 1 is a diagram for facilitating understanding of the overview of the genetic characteristic estimation device 2000, and the operation of the genetic characteristic estimation device 2000 is not limited to that illustrated in FIG. 1.

The genetic characteristic estimation device 2000 computes an index value related to a genetic characteristic of a target organism 10 (hereinafter, referred to as genetic characteristic index value). The target organism 10 is any organism that is a target of computation of the genetic characteristic index value c, and may be a human or other animal, or may be a plant.

The genetic characteristic is a characteristic that appears due to the effect of a gene. For example, the genetic characteristic is a characteristic related to a disease, such as a high probability of contracting a disease or a speed of progression of a disease. In another example, the genetic characteristic is a physical characteristic such as a height or a weight. In another example, the genetic characteristic is a degree of efficiency of a medicine, such as the degree of resistance or sensitivity to a medicine.

The genetic characteristic index value is, for example, a polygenic risk score. However, the genetic characteristic index value may be any index value representing the genetic characteristic of the target organism 10, and is not limited to the polygenic risk score.

In order to compute the genetic characteristic index value of the target organism 10, the genetic characteristic estimation device 2000 acquires genetic mutation information 30 and position information 40. The genetic mutation information 30 indicates information regarding a genetic mutation in a deoxyribonucleic acid (DNA) sequence of a cell 20 (target cell 20) obtained from the target organism 10. It is noted that, the genetic mutation information 30 indicates at least a position in the DNA sequence for each of one or more genetic mutations of the target cell 20.

The position information 40 is information that associates the position in the DNA sequence with the type of the cell or organ. For example, the position information 40 indicates, for each type of cell or organ, the position in the DNA sequence to be particularly focused on for computing the genetic characteristic index value.

Examples of the type of the cell include a nerve cell, a glial cell, a blood cell, and a skin cell. It is noted that the granularity of classification is arbitrary. For example, the glial cell may be further subdivided, and more specific types such as microglia and oligodendrocyte may be used.

Examples of the type of the organ include a brain, a heart, and a lung. However, the granularity of classification is also arbitrary for the type of the organ. For example, a group including a plurality of types of organs such as a “respiratory system” may be used as the type of the organ.

From genetic mutations indicated by the genetic mutation information 30, the genetic characteristic estimation device 2000 determines a genetic mutation that is at a position associated, by the position information 40, with the type of the target cell 20 or the type of the organ having the target cell 20. Hereinafter, the genetic mutation determined here is referred to as “mutation of interest”. The genetic characteristic estimation device 2000 computes the genetic characteristic index value for the target organism 10 based on the characteristic of the mutation of interest.

It is noted that, among the genetic mutations of the target cell 20, characteristics of genetic mutations other than the mutation of interest may not be used or may be used for computation of the genetic characteristic index value. However, in the latter case, the characteristic of the mutation of the interest has higher influence on the genetic characteristic index value (contribution to the genetic characteristic index value) than the characteristic of the mutation other than that of the mutation of the interest. A specific method thereof will be described later.

### Example of Advantageous Effect

With the genetic characteristic estimation device 2000 of the present example embodiment, the genetic mutation (the mutation of interest) at the position associated with the type of the target cell 20 or the type of the organ having the target cell 20 by the position information 40 is determined from the genetic mutations of the target cell 20 of the target organism 10. Then, an index value regarding the genetic characteristic of the target organism 10 is computed based on the characteristic of the mutation of interest. The characteristics of genetic mutations other than the mutation of interest are not used for computation of the genetic characteristic index value, or are used in such a way as to have a lower influence on the genetic characteristic index value than the characteristic of the mutation of interest.

According to such a way, it is possible to compute the genetic characteristic index value so as to represent the genetic characteristic of the target organism 10 more accurately than that of a case where the mutation of interest and other genetic mutations are not distinguished. For example, in the position information 40, the type of the target cell 20 or the type of the organ having the target cell 20 is associated with the position on the DNA sequence considered to have a high influence on the genetic characteristic. As a result, in the computation of the genetic characteristic index value, attention is paid to the characteristic of the genetic mutation at the position considered to have a high influence on the genetic characteristic. Therefore, it is possible to compute the genetic characteristic index value so as to represent the genetic characteristic of the target organism 10 more accurately than that of a case where such attention is not made.

Hereinafter, the genetic characteristic estimation device 2000 of the present example embodiment will be described in more detail.

### <Example of Functional Configuration>

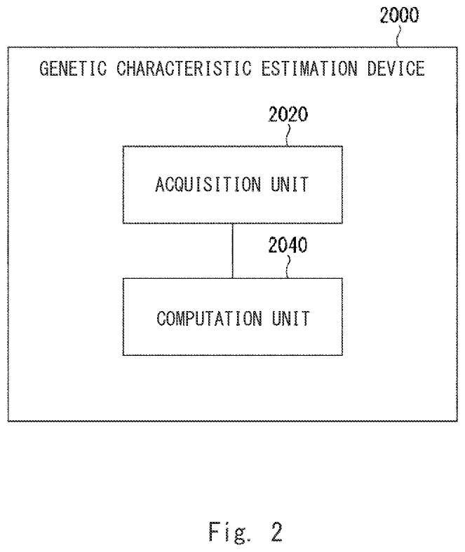

FIG. 2 is a block diagram illustrating a functional configuration of the genetic characteristic estimation device 2000 of the first example embodiment. The genetic characteristic estimation device 2000 includes an acquisition unit 2020 and a computation unit 2040. The acquisition unit 2020 acquires the genetic mutation information 30 and the position information 40 for the target cell 20 of the target organism 10. The computation unit 2040 determines a genetic mutation at a position that the position information 40 associates with the type of the target cell 20 or the type of the organ having the target cell 20, from the genetic mutations indicated by the genetic mutation information 30. Then, the computation unit 2040 computes the genetic characteristic index value based on the characteristic of the determined genetic mutation.

### <Example of Hardware Configuration>

Each functional component of the genetic characteristic estimation device 2000 may be implemented by hardware (for example, a hard-wired electronic circuit or the like) that implements each functional configuration unit, or may be implemented by a combination of hardware and software (for example, a combination of an electronic circuit and a program that controls the electronic circuit or the like). Hereinafter, a case where each functional component of the genetic characteristic estimation device 2000 is implemented by a combination of hardware and software will be further described.

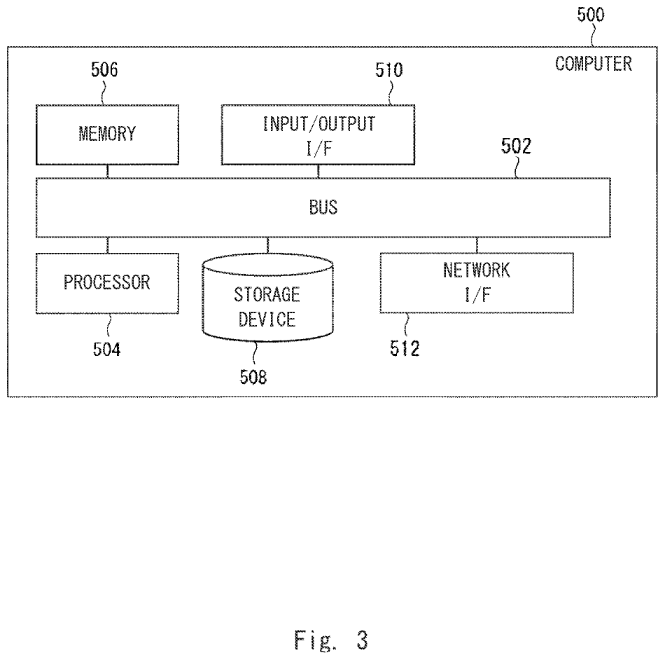

FIG. 3 is a block diagram illustrating a hardware configuration of a computer 500 that implements the genetic characteristic estimation device 2000. The computer 500 is any computer. For example, the computer 500 is a stationary computer such as a personal computer (PC) or a server machine. In another example, the computer 500 is a portable computer such as a smartphone or a tablet terminal. The computer 500 may be a special-purpose dedicated computer designed to implement the genetic characteristic estimation device 2000, or may be a general-purpose computer.

For example, by installing a predetermined application in the computer 500, each function of the genetic characteristic estimation device 2000 is implemented in the computer 500. The above-described application is configured by a program for implementing the functional components of the genetic characteristic estimation device 2000. Note that a method for acquiring the program is arbitrary. For example, the program can be acquired from a storage medium (a DVD disk, a USB memory, or the like) in which the program is stored. In another example, the program can be acquired by downloading the program from a server device that manages a storage unit in which the program is stored.

The computer 500 has a bus 502, a processor 504, a memory 506, a storage device 508, an input/output interface 510, and a network interface 512. The bus 502 is a data transmission path for the processor 504, the memory 506, the storage device 508, the input/output interface 510, and the network interface 512 to transmit and receive data to and from each other. However, the method for connecting the processor 504 and the like to each other is not limited to the bus connection.

The processor 504 is various processors such as a central processing unit (CPU), a graphics processing unit (GPU), and a field-programmable gate array (FPGA). The memory 506 is a main storage device implemented by using a random access memory (RAM) or the like. The storage device 508 is an auxiliary storage device implemented by using a hard disk, a solid state drive (SSD), a memory card, or a read only memory (ROM).

The input/output interface 510 is an interface for connecting the computer 500 and an input/output device. For example, an input device such as a keyboard and an output device such as a display device are connected to the input/output interface 510.

The network interface 512 is an interface connecting the computer 500 to a network. The network may be a local area network (LAN), or may be a wide area network (WAN).

The storage device 508 stores a program (program for implementing the above-described application) for implementing each functional component of the genetic characteristic estimation device 2000. The processor 504 reads the program to the memory 506 and executes the program to implement each functional component of the genetic characteristic estimation device 2000.

The genetic characteristic estimation device 2000 may be implemented by one computer 500 or may be implemented by a plurality of computers 500. In the latter case, the configurations of the computers 500 do not need to be the same, and can be different from each other.

### <Flow of Processing>

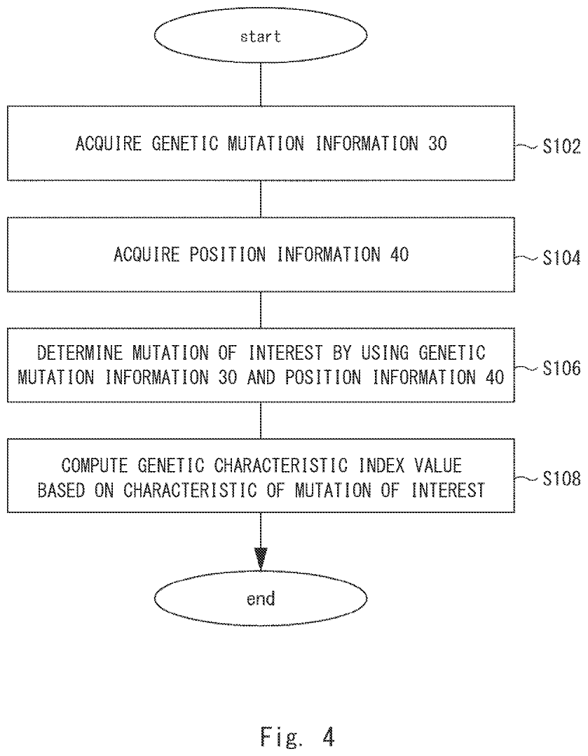

FIG. 4 is a flowchart illustrating a flow of processes executed by the genetic characteristic estimation device 2000 of the first example embodiment. The acquisition unit 2020 acquires the genetic mutation information 30 (S102). The acquisition unit 2020 acquires the position information 40 (S104). The computation unit 2040 determines the mutation of interest by using the genetic mutation information 30 and the position information 40 (S106). Specifically, the computation unit 2040 determines, as the mutation of interest, a genetic mutation that the position information 40 associates with the type of the target cell 20 or the type of the organ having the target cell 20, from the genetic mutations indicated by the genetic mutation information 30. Then, the computation unit 2040 computes the genetic characteristic index value based on the degree of contribution of the mutation of interest to the genetic characteristic (S108).

It is noted that the flow of processes illustrated in FIG. 4 is an example of the flow of processes executed by the genetic characteristic estimation device 2000, and the flow of processes executed by the genetic characteristic estimation device 2000 is not limited to that illustrated in FIG. 4. For example, the acquisition of the genetic mutation information 30 (S102) and the acquisition of the position information 40 (S104) may be performed in the order opposite to the above-described order, or may be performed in parallel with each other.

### <Acquisition of Genetic Mutation Information 30: S102>

The acquisition unit 2020 acquires the genetic mutation information 30 (S102). As described above, the genetic mutation information 30 indicates, for a target cell 20, information regarding a genetic mutation in the DNA sequence of that target cell 20. The genetic mutation information 30 indicates at least the position of each genetic mutation of the target cell 20 in the DNA sequence of the target cell 20.

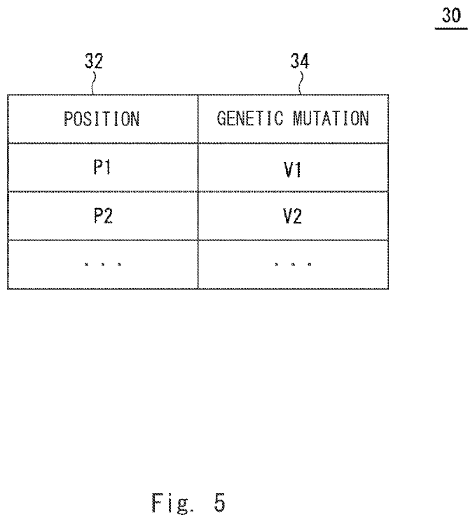

FIG. 5 is a diagram illustrating the genetic mutation information 30 in a table format. The genetic mutation information 30 in FIG. 5 has two columns of a position 32 and a genetic mutation 34. The position 32 indicates the position of the target cell 20 in the DNA sequence. The genetic mutation 34 indicates the genetic mutation of the target cell 20 at the position in the DNA sequence indicated by the corresponding position 32. For example, a record in the first row of FIG. 5 indicates that the target cell 20 has a genetic mutation VI at a position P1.

There are various ways for the acquisition unit 2020 to acquire the genetic mutation information 30. For example, the genetic mutation information 30 is stored in advance in a storage unit accessible from the genetic characteristic estimation device 2000. In this case, the acquisition unit 2020 accesses the storage unit to acquire the genetic mutation information 30. As a more specific example, in a case where the target organism 10 is a patient in a hospital, the genetic mutation information 30 can be included in data representing a medical record of the target organism 10 (so-called electronic medical record). In this case, the acquisition unit 2020 acquires the genetic mutation information 30 of the target organism 10 from the electronic medical record of the target organism 10. It is noted that an existing technique can be used as a technique for acquiring desired information from an electronic medical record of a specific person. In another example, the genetic mutation information 30 may be transmitted from another device to the genetic characteristic estimation device 2000.

### <Acquisition of Position Information 40: S104>

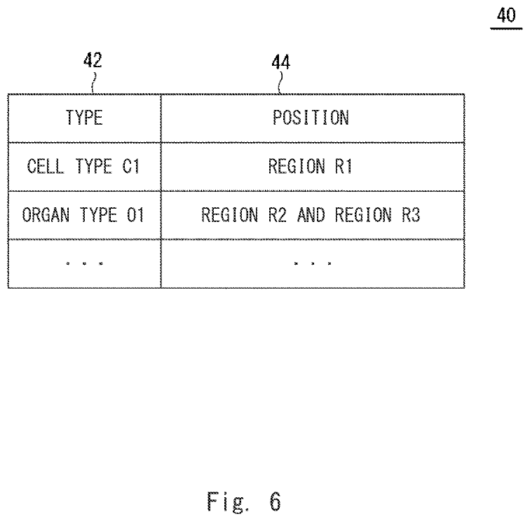

The acquisition unit 2020 acquires the position information 40 (S104). As described above, the position information 40 is information in which the position in the DNA sequence is associated with the type of the cell or organ. FIG. 6 is a diagram illustrating the position information 40 in a table format. The position information 40 has two columns of a type 42 and a position 44. The type 42 indicates the type of the cell or organ. The position 44 indicates one or more positions in the DNA sequence. In a case where the position 44 indicates more than one position, the position 44 may indicate a particular region in the DNA sequence. For example, in a record in the first row of FIG. 6, a range R1 in the DNA sequence is associated with a cell type C1.

Specific examples of the region in the DNA sequence include a promoter, an enhancer, a chemically modified region (a region where DNA methylation has occurred), and a specific gene. These regions directly or indirectly affect gene expression and a protein structure. Therefore, it can be argued that the genetic mutations in these regions have a higher influence on the genetic characteristic of the organism than the genetic mutations in other regions. Therefore, it is possible to more accurately ascertain the genetic characteristic of the target organism 10 by particularly focusing on the genetic mutations in these regions among the genetic mutations of the target organism 10.

There are various ways for the acquisition unit 2020 to acquire the position information 40. For example, the position information 40 is stored in advance in the storage unit accessible from the genetic characteristic estimation device 2000. In this case, the acquisition unit 2020 accesses the storage unit to acquire the position information 40. In another example, the position information 40 may be transmitted from another device to the genetic characteristic estimation device 2000.

The position information 40 may be prepared for each type of genetic characteristic for which the genetic characteristic index value is to be computed. For example, in this case, pieces of position information 40 different from each other are used in the computation of the genetic characteristic index value representing a risk of lung cancer and in the computation of the genetic characteristic index value representing a risk of Alzheimer's disease.

It is noted that, a plurality of pieces of position information 40 may be prepared for one type of genetic characteristic. In this case, the genetic characteristic estimation device 2000 may compute one genetic characteristic index value by using a plurality of pieces of position information 40, or may compute the genetic characteristic index value individually for each piece of position information 40. By computing the genetic characteristic index values individually for each piece of position information 40, it is possible to evaluate a risk or the like regarding the target organism 10 for each type of organ or cell for one genetic characteristic.

Suppose that, by computing the genetic characteristic index value representing a risk of suffering from schizophrenia for each of three types of organs including the brain, the liver, and the intestine for a patient with schizophrenia, an organ highly related to schizophrenia of the patient is predicted from these organs. In this case, the position information 40 of “type 42=brain”, the position information 40 of “type 42=liver”, and the position information 40 of “type 42=intestine” are prepared. Then, the genetic characteristic estimation device 2000 individually computes the genetic characteristic index value for each of the three pieces of position information 40.

Suppose that both the genetic characteristic index values for the brain and the intestine indicate that the risk of suffering from schizophrenia is high, while the genetic characteristic index value for the liver indicates that the risk of suffering from schizophrenia is not high. In this case, it can be ascertained that it is highly likely that the brain and the intestine are related to schizophrenia of the patient.

In a case where the position information 40 is determined for each type of genetic characteristic, for example, the genetic characteristic estimation device 2000 acquires information designating for which type of genetic characteristic the genetic characteristic index value is to be computed (the type of the genetic characteristic for which the genetic characteristic index value is to be computed). For example, this information is input by a user. In this case, the acquisition unit 2020 acquires the position information 40 corresponding to the type of the genetic characteristic designated by the user.

### <Determination of Mutation of Interest: S106>

The computation unit 2040 determines a position that the position information 40 associates with the type of the target cell 20 or the type of the organ having the target cell 20, and determines a genetic mutation that the genetic mutation information 30 indicates for that position as the mutation of interest (S106). For example, the computation unit 2040 determines a record whose type 42 indicates the type of the target cell 20 or the type of the organ having the target cell 20 from the position information 40.

The computation unit 2040 determines, from the records of the genetic mutation information 30, a record whose position 32 indicates a position that is indicated by the position 44 of the determined record of the position information 40. Then, the computation unit 2040 determines a genetic mutation indicated by the genetic mutation 34 of the determined record of the genetic mutation information 30 as the mutation of interest.

Suppose that the record of the position information 40 whose type 42 indicates the type of the target cell 20 indicates two data of “promoter” and “enhancer” at the position 44. In this case, the computation unit 2040 determines a record whose position 32 indicates a position included in the promoter or the enhancer from the genetic mutation information 30. Then, the computation unit 2040 determines the genetic mutation indicated by the genetic mutation 34 of the determined record as the mutation of interest.

It is noted that, it may be predefined or dynamically determined by a user which one of the cell type and the organ type is to be used or both of them are to be used. In the latter case, for example, the genetic characteristic estimation device 2000 provides, to the user of the genetic characteristic estimation device 2000, an input interface (for example, an input screen) through which it is possible to select, from the cell type and the organ type, one to be used to determine the mutation of interest. Then, the genetic characteristic estimation device 2000 determines the mutation of interest based on a user input result. Suppose that the user selects “cell type”. In this case, the computation unit 2040 determines a position that the position information 40 associates with the type of the target cell 20.

### <Computation of Genetic Characteristic Index Value: S108>

The computation unit 2040 computes the genetic characteristic index value based on the characteristic of the mutation of interest (S108). For example, the computation unit 2040 computes a score based on the characteristic for each mutation of interest. Then, the genetic characteristic index value is computed based on the score computed for each mutation of interest.

For example, a computational formula for computing the score from the characteristic of the genetic mutation and a computational formula for computing the genetic characteristic index value based on the score computed for each mutation of interest are determined in advance. For example, these computational formulas are expressed by the following equation (1).

$$Equation 1$$

$$S = g ( \{ h ( f [ i ] ) \vert i ∈ A \} ) \qquad ( 1 )$$

It is noted that, S represents the genetic characteristic index value. A represents a set of mutations of interest. i represents an identifier of the genetic mutation. Hereinafter, a genetic mutation whose identifier is i is described as the genetic mutation i. f[i] represents the characteristic of the genetic mutation i. h( ) is the computational formula for computing the score from the characteristic of the genetic mutation. g( ) is the computational formula for computing the genetic characteristic index value based on the scores computed for the respective mutations of interest.

For example, the genetic characteristic index value is computed as a simple sum or a weighted sum of the scores computed for the respective mutations of interest. In this case, the equation (1) can be expressed as the following equation (2).

$$Equation 2$$

$$S = {\sum }_{i ∈ A} α [ i ] * h ( f [ i ] ) \qquad ( 2 )$$

It is noted that α[i] is a weight given to the genetic mutation i. For example, the weight of the genetic mutation i is determined according to the position of the target cell 20 in the DNA sequence. If α[i]=1 is set for all i, the equation (2) is a simple sum.

There are various possible ways to convert the characteristic of the mutation of interest into a score. For example, the number of specific alleles that the mutation of interest has is utilized as the score. In another example, the strength of a correlation (e.g., linkage disequilibrium or the like) between the mutation of interest and a mutation in the vicinity in the DNA, or the strength of the activity of the promoter or the enhancer is also used as the score.

It is noted that the magnitude of the influence of the characteristic of the genetic mutation on the genetic characteristic may vary depending on the type of the genetic characteristic. For example, it is highly likely that, regarding a certain genetic mutation, the magnitude of the influence on the risk of lung cancer, the magnitude of the influence on the risk of Alzheimer's disease, and the magnitude of the influence on the ease of growth of height are different from each other. Therefore, it is preferable that a computational formula for computing the score from the characteristic of the genetic mutation is determined for each type of genetic characteristic.

In a case where the computational formula for computing the score from the characteristic of the genetic mutation is defined for each type of genetic characteristic, for example, the genetic characteristic estimation device 2000 acquires information designating the type of the genetic characteristic for which the genetic characteristic index value is to be computed. As described above, for example, this information is input by the user. The computation unit 2040 computes the genetic characteristic index value by using a computational formula corresponding to the designated type of genetic characteristic, among computational formulas prepared in advance. Similarly, the computational formula for computing the genetic characteristic index value based on the scores computed for respective mutations of interest may also be determined for each genetic characteristic for which the genetic characteristic index value is to be computed.

### <<Use of Genetic Mutation Other Than Mutation of Interest>>

It is noted that, as described above, the characteristic of a genetic mutation other than the mutation of interest may be used to compute the genetic characteristic index value. In this case, a computational formula for computing the genetic characteristic is expressed as, for example, the following equation (3).

$$Equation 3$$

$$S = g ( \{ h ( f [ i ] ) \vert i ∈ A \} , \{ h ( f [ j ] ) \vert j ∈ B - A \} ) \qquad ( 3 )$$

It is noted that, the set B is a set of all genetic mutations contained in the target cell 20. Therefore, the set B-A represents a set of genetic mutations other than the mutation of interest among the genetic mutations contained in the target cells 20. j is an identifier of the genetic mutation included in the set B.

In addition, in a case where the genetic characteristic index value is computed as a simple sum, a weighted sum, or the like of the scores computed for the respective mutations of interest, the equation (3) can be expressed as the following equation (4).

$$Equation 4$$

$$S = {\sum }_{i ∈ A} α [ i ] * h ( f [ i ] ) + {\sum }_{j ∈ B - A} β [ i ] * h ( f [ j ] ) \\ s . t . ∀ i ∈ A , ∀ j ∈ B - A , α [ i ] > β [ j ] \qquad ( 4 )$$

In the equation (4), the constraint “α[i]>β[j]” is one of the ways to implement the constraint that “the magnitude of the influence of the characteristic of the mutation of interest on the genetic characteristic index value is larger than that of the influence of the characteristic of a genetic mutation other than the mutation of interest on the genetic characteristic index value”. However, the way of implementing the constraint is not limited to the way of defining “α[i]>β[j]”.

### <<Consideration of Degree of Contribution>>

In the computation of the genetic characteristic index value, it is possible to take into consideration the degree of contribution of each genetic mutation to the genetic characteristic. In this case, for example, the computation unit 2040 selects a genetic mutation to be used for computation of the genetic characteristic index value, based on the degree of contribution of each genetic mutation to the genetic characteristic. More specifically, the computation unit 2040 selects genetic mutations whose degree of contribution to the genetic characteristic is equal to or higher than a threshold from the genetic mutations included in the target cell 20, and computes the genetic characteristic index value based on the characteristics of the selected genetic mutations. it is possible to compute the genetic characteristic index value that more accurately represents the genetic characteristic of the target organism 10, by selecting the genetic mutation to be used for the computation of the genetic characteristic index value based on the degree of contribution to the genetic characteristic as described above.

In a case where only the mutation of interest is used and the degree of contribution is taken into consideration, the computational formula for computing the genetic characteristic index value can be expressed as, for example, the following equation (5).

$$Equation 5$$

$$S = g ( \{ h ( f [ i ] ) \vert i ∈ A , c [ i ] \geq th \} ) \qquad ( 5 )$$

It is noted that, c[i] represents the degree of contribution of the genetic mutation i to the genetic characteristic. th represents the threshold of the degree of contribution to be used to select genetic mutations. In this example, only genetic mutations whose degree of contribution is equal to or larger than th are used for computation of the genetic characteristic index value.

In a case where both the mutation of interest and other genetic mutations are used and the degree of contribution is taken into consideration, the computational formula for computing the genetic characteristic index value can be expressed as, for example, the following equation (6).

$$Equation 6$$

$$S = g ( \{ h ( f [ i ] ) \vert i ∈ A , c [ i ] \geq th \} , \{ h ( f [ j ] ) \vert j ∈ B - A , c [ j ] \geq th \} ) \qquad ( 6 )$$

It is noted that, in the equation (6), for both the mutation of interest and other genetic mutations, only genetic mutations whose degree of contribution is equal to or higher than the threshold are selected. However, the computation unit 2040 does not have to perform the selection based on the degree of contribution for the mutation of interest, and may perform the selection based on the degree of contribution only for genetic mutations other than the mutation of interest. In this case, the mutation of interest is used for computation of the genetic characteristic index value regardless of the degree of contribution. On the other hand, regarding genetic mutations other than the mutation of interest, only genetic mutations whose degree of contribution is equal to or higher than the threshold are used for computation of the genetic characteristic index value. In this case, a formula for computing the genetic characteristic index value can be expressed as the following equation (7).

$$Equation 7$$

$$S = g ( \{ h ( f [ i ] ) \vert i ∈ A \} , \{ h ( f [ j ] ) \vert j ∈ B - A , c [ j ] \geq th \} ) \qquad ( 7 )$$

Unlike the equation (6), the equation (7) does not include the condition that c[i]>=th. Therefore, the mutation of interest is used for computation of the genetic characteristic index value regardless of the degree of contribution thereof. In order to consider the degree of contribution of each genetic mutation to the genetic characteristic as described above, for example, the computation unit 2040 acquires information indicating the degree of contribution of the genetic mutation to the genetic characteristic (hereinafter, contribution degree information). The contribution degree information is stored in advance in an arbitrary storage unit in a manner that it can be acquired from the genetic characteristic estimation device 2000. The computation unit 2040 acquires the contribution degree information for the genetic characteristic for which the genetic characteristic index value is to be computed, and uses the acquired contribution degree information to select the genetic mutation to be used for the computation of the genetic characteristic index value.

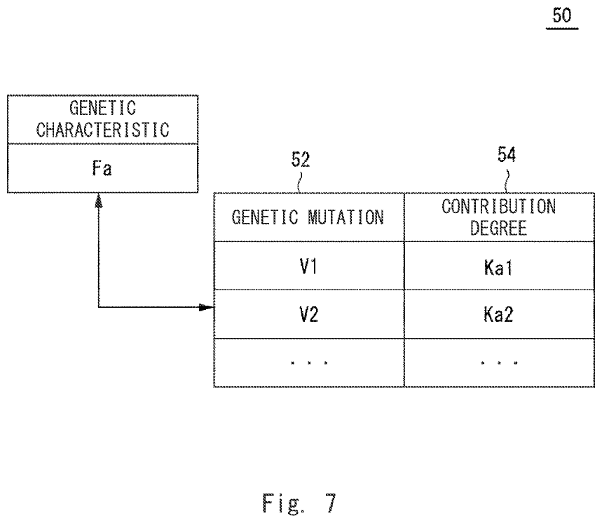

FIG. 7 is a diagram illustrating the contribution degree information in a table format. Contribution degree information 50 in FIG. 7 has two columns of a genetic mutation 52 and the contribution degree 54. The genetic mutation 52 indicates identification information of a genetic mutation. The contribution degree 54 indicates the degree of contribution of the genetic mutation indicated by the corresponding genetic mutation 52 to the genetic characteristic.

The contribution degree information 50 is prepared for each type of genetic characteristic. For example, the contribution degree information 50 in FIG. 7 indicates the degree of contribution of each genetic mutation to a genetic characteristic of a type Fa. Therefore, for example, a record in the first row of the contribution degree information 50 in FIG. 7 indicates that the degree of contribution of a genetic mutation VI to the genetic characteristic Fa is Ka1.

The contribution degree information may be provided for each of some indexes for an organism (such as a blood glucose level or a brain volume) that may affect the genetic characteristic. Specifically, the contribution degree information 50 indicating a higher degree of contribution for a genetic mutation whose correlation with a specific index is stronger is prepared. When computing the genetic characteristic index value for a genetic characteristic related to a specific index, the genetic characteristic estimation device 2000 uses the contribution degree information 50 generated based on the strength of the correlation with the index.

For example, the strength of the correlation with the blood glucose level is examined for each genetic mutation, and the contribution degree information 50 is generated so as to show a higher contribution degree of a genetic mutation as the correlation of that genetic mutation with the blood glucose level is stronger. The genetic characteristic estimation device 2000 uses the contribution degree information 50 when computing the genetic characteristic index value (for example, a risk of diabetes) for a disease related to the blood glucose level.

In a case of using the contribution degree information 50 prepared for each index, information that associates a genetic characteristic with an index related to the genetic characteristic is prepared in advance. For example, in this information, the index of “blood glucose level” is associated with the genetic characteristic of “risk of diabetes”. When computing the genetic characteristic index value for a certain genetic characteristic, the genetic characteristic estimation device 2000 uses, in addition to the mutation of interest, a genetic mutation whose degree of contribution is equal to or higher than the threshold in the contribution degree information 50 for an index associated with that genetic characteristic.

### <Output by Genetic Characteristic Estimation Device 2000>

The genetic characteristic estimation device 2000 outputs information indicating the genetic characteristic index value (hereinafter, output information). For example, the output information includes a type of genetic characteristic and a genetic characteristic index value computed for the type of genetic characteristic. In another example, the output information may include various types of information used for computing the genetic characteristic index value. Examples of the information used for computing the genetic characteristic index value include the position (a promoter, an enhancer, or the like) that the position information 40 associates with the type of the target cell 20 or the type of the organ having the target cell 20, the mutation of interest determined by the computation unit 2040, and the like. Furthermore, in a case where the genetic mutation is selected based on the threshold of the degree of contribution, the output information may further include information such as the selected genetic mutation and the threshold of the degree of contribution.

The output information is output in an arbitrary manner. For example, the genetic characteristic estimation device 2000 puts the output information in an arbitrary storage unit accessible from the genetic characteristic estimation device 2000. In another example, the genetic characteristic estimation device 2000 causes any display device accessible from the genetic characteristic estimation device 2000 to display the output information. In another example, the genetic characteristic estimation device 2000 transmits the output information to any device accessible from the genetic characteristic estimation device 2000.

Although the present invention has been described above with reference to the example embodiments, the present invention is not limited to the above-described example embodiments. Various changes that can be understood by those skilled in the art can be made to the configurations and details of the present invention within the scope of the present invention.

Note that, in the above-described example, the program can be stored and provided to a computer using any type of non-transitory computer-readable media. The non-transitory computer-readable media include various types of tangible storage media. Examples of the non-transitory computer-readable media include magnetic storage media (such as floppy disks, magnetic tapes, hard disk drives, or the like), optical magnetic storage media (for example, magneto-optical disks), CD-ROM, CD-R, CD-R/W, and semiconductor memories (such as mask ROM, PROM (Programmable ROM), EPROM (Erasable PROM), flash ROM, RAM, or the like). Further, programs may be provided to computers by various types of transitory computer-readable media. Examples of the transitory computer-readable media include electric signals, optical signals, and electromagnetic waves. The transitory computer readable media can supply the programs to the computer via wired or wireless communication paths such as wires and optical fiber.

Some or all of the above-described example embodiments can be described as in the following supplementary notes, but are not limited to the following supplementary notes.

### (Supplementary Note 1)

A genetic characteristic estimation device comprising:
an acquisition unit configured to acquire genetic mutation information regarding a genetic mutation in a deoxyribonucleic acid (DNA) sequence of a target cell obtained from a target organism, and position information that associates a position in the DNA sequence with a type of a cell or a type of an organ; and
a computation unit configured to determine a mutation of interest, which is a genetic mutation at a position that the position information associates with the type of the target cell or the type of the organ having the target cell, from the genetic mutations indicated by the genetic mutation information, and compute a genetic characteristic index value, which represents a genetic characteristic of the target organism, based on a characteristic of the mutation of interest.

### (Supplementary Note 2)

The genetic characteristic estimation device according to supplementary note 1,
wherein the position indicated by the position information represents a promoter, an enhancer, a chemically modified region, or a region of a specific gene in a DNA that the cell associated therewith or a cell of the organ associated therewith has.

### (Supplementary Note 3)

The genetic characteristic estimation device according to supplementary note 1 or 2,
wherein the computation unit performs:
   acquiring contribution degree information that indicates a contribution degree, which is a magnitude of contribution of each genetic mutation to the genetic characteristic; and
   computing the genetic characteristic index value based on the characteristic of the mutation of interest whose contribution degree is equal to or higher than a threshold.

### (Supplementary Note 4)

The genetic characteristic estimation device according to supplementary note 1 or 2,
wherein the computation unit performs:
   computing a first score based on the characteristic of the mutation of interest and a second score based on a characteristic of a genetic mutation other than the mutation of interest:
   assigning different weights to the first score and the second score in such a way that an influence of the first score on the genetic characteristic index value is higher than an influence of the second score on the genetic characteristic index value; and
   computing the genetic characteristic index value by using the first score and the second score to which the weights are assigned.

### (Supplementary Note 5)

The genetic characteristic estimation device according to supplementary note 4,
wherein the computation unit performs:
   acquiring contribution degree information that indicates a contribution degree, which is a magnitude of contribution of each genetic mutation to a genetic characteristic; and
   computing the second score only for a genetic mutation whose contribution degree is equal to or higher than a threshold among genetic mutations other than the mutation of interest.

### (Supplementary Note 6)

The genetic characteristic estimation device according to supplementary note 5,
wherein the computation unit computes the first score only for the mutation of interest whose contribution degree is equal to or higher than the threshold.

### (Supplementary Note 7)

The genetic characteristic estimation device according to any one of supplementary notes 1 to 6, wherein the genetic characteristic index value is a polygenic risk score.

### (Supplementary Note 8)

A control method executed by a computer, the control method comprising:
an acquisition step of acquiring genetic mutation information regarding a genetic mutation in a deoxyribonucleic acid (DNA) sequence of a target cell obtained from a target organism, and position information that associates a position in the DNA sequence with a type of a cell or a type of an organ; and
a computation step of determining a mutation of interest, which is a genetic mutation at a position that the position information associates with the type of the target cell or the type of the organ having the target cell, from the genetic mutations indicated by the genetic mutation information, and computing a genetic characteristic index value, which represents a genetic characteristic of the target organism, based on a characteristic of the mutation of interest.

### (Supplementary Note 9)

The control method according to supplementary note 8,
wherein the position indicated by the position information represents a promoter, an enhancer, a chemically modified region, or a region of a specific gene in a DNA that the cell associated therewith or a cell of the organ associated therewith has.

### (Supplementary Note 10)

The control method according to supplementary note 8 or 9,
wherein in the computation step:
   acquiring contribution degree information that indicates a contribution degree, which is a magnitude of contribution of each genetic mutation to the genetic characteristic; and
   computing the genetic characteristic index value based on the characteristic of the mutation of interest whose contribution degree is equal to or higher than a threshold.

### (Supplementary Note 11)

The control method according to supplementary note 8 or 9,
wherein in the computation step:
   computing a first score based on the characteristic of the mutation of interest and a second score based on a characteristic of a genetic mutation other than the mutation of interest:
   assigning different weights to the first score and the second score in such a way that an influence of the first score on the genetic characteristic index value is larger than an influence of the second score on the genetic characteristic index value; and
   computing the genetic characteristic index value by using the first score and the second score to which the weights are assigned.

### (Supplementary Note 12)

The control method according to supplementary note 11,
wherein in the computation step:
   acquiring contribution degree information that indicates a contribution degree, which is a magnitude of contribution of each genetic mutation to the genetic characteristic; and
   computing the second score only for a genetic mutation whose contribution degree is equal to or higher than a threshold among genetic mutations other than the mutation of interest.

### (Supplementary Note 13)

The control method according to supplementary note 12,
wherein in the computation step, computing the first score only for the mutation of interest whose contribution degree is equal to or higher than the threshold.

### (Supplementary Note 14)

The control method according to any one of supplementary notes 8 to 13, wherein the genetic characteristic index value is a polygenic risk score.

### (Supplementary Note 15)

A non-transitory computer-readable medium storing a program for causing a computer to execute:
an acquisition step of acquiring genetic mutation information regarding a genetic mutation in a deoxyribonucleic acid (DNA) sequence of a target cell obtained from a target organism, and position information that associates a position in the DNA sequence with a type of a cell or a type of an organ; and
a computation step of determining a mutation of interest, which is a genetic mutation at a position that the position information associates with the type of the target cell or the type of the organ having the target cell, from the genetic mutations indicated by the genetic mutation information, and computing a genetic characteristic index value, which represents a genetic characteristic of the target organism, based on a characteristic of the mutation of interest.

### (Supplementary Note 16)

The computer-readable medium according to supplementary note 15, wherein the position indicated by the position information represents a promoter, an enhancer, a chemically modified region, or a region of a specific gene in a DNA that the cell associated therewith or a cell of the organ associated therewith has.

### (Supplementary Note 17)

The computer-readable medium according to supplementary note 15 or 16,
wherein in the computation step:
   acquiring contribution degree information that indicates a contribution degree, which is a magnitude of contribution of each genetic mutation to the genetic characteristic; and
   computing the genetic characteristic index value based on the characteristic of the mutation of interest whose contribution degree is equal to or higher than a threshold.

### (Supplementary Note 18)

The computer-readable medium according to supplementary note 15 or 16,
wherein in the computation step:
   computing a first score based on the characteristic of the mutation of interest and a second score based on a characteristic of a genetic mutation other than the mutation of interest:
   assigning different weights to the first score and the second score in such a way that an influence of the first score on the genetic characteristic index value is larger than an influence of the second score on the genetic characteristic index value; and
   computing the genetic characteristic index value by using the first score and the second score to which the weights are assigned.

### (Supplementary Note 19)

The computer-readable medium according to supplementary note 18,
wherein in the computation step:
   acquiring contribution degree information that indicates a contribution degree, which is a magnitude of contribution of each genetic mutation to the genetic characteristic; and
   computing the second score only for a genetic mutation whose contribution degree is equal to or higher than a threshold among genetic mutations other than the mutation of interest.

### (Supplementary Note 20)

The computer-readable medium according to supplementary note 19,
wherein in the computation step, computing the first score only for the mutation of interest whose contribution degree is equal to or higher than the threshold.

### (Supplementary Note 21)

The computer-readable medium according to any one of supplementary notes 15 to 20, wherein the genetic characteristic index value is a polygenic risk score.

### REFERENCE SIGNS LIST

10 TARGET ORGANISM
20 TARGET CELL
30 GENETIC MUTATION INFORMATION
32 POSITION
34 GENETIC MUTATION
40 POSITION INFORMATION
42 TYPE
44 POSITION
50 CONTRIBUTION DEGREE INFORMATION
52 GENETIC MUTATION
54 CONTRIBUTION DEGREE
500 COMPUTER
502 BUS
504 PROCESSOR
506 MEMORY
508 STORAGE DEVICE
510 INPUT/OUTPUT INTERFACE
512 NETWORK INTERFACE
2000 GENETIC CHARACTERISTIC ESTIMATION DEVICE
2020 ACQUISITION UNIT
2040 COMPUTATION UNIT

1. A genetic characteristic estimation device comprising:
at least one memory that is configured to store instructions; and
at least one processor that is configured to execute the instructions to:
acquire genetic mutation information and position information, the genetic mutation information indicating a genetic mutation in a deoxyribonucleic acid (DNA) sequence of a target cell obtained from a target organism, the position information associating a position in the DNA sequence with a type of a cell or a type of an organ;
determine a mutation of interest, which is a genetic mutation at a position that the position information associates with the type of the target cell or the type of the organ having the target cell, from the genetic mutations indicated by the genetic mutation information; and
compute a genetic characteristic index value based on a characteristic of the mutation of interest, the genetic characteristic index value representing a genetic characteristic of the target organism.

2. The genetic characteristic estimation device according to claim 1,
wherein the position indicated by the position information represents a promoter, an enhancer, a chemically modified region, or a region of a specific gene in a DNA that the cell associated with that position has or a cell of the organ associated with that position has.

3. The genetic characteristic estimation device according to claim 1,
wherein the computation of the genetic characteristic index value includes:
acquiring contribution degree information that indicates a contribution degree, which is a magnitude of contribution of each genetic mutation to the genetic characteristic; and
computing the genetic characteristic index value based on the characteristic of the mutation of interest whose contribution degree is equal to or higher than a threshold.

4. The genetic characteristic estimation device according to claim 1,
wherein the computation of the genetic characteristic index value includes:
computing a first score based on the characteristic of the mutation of interest and a second score based on a characteristic of a genetic mutation other than the mutation of interest;
assigning different weights to the first score and the second score in such a way that an influence of the first score on the genetic characteristic index value is higher than an influence of the second score on the genetic characteristic index value; and
computing the genetic characteristic index value by using the first score and the second score to which the weights are assigned.

5. The genetic characteristic estimation device according to claim 4,
wherein the computation of the genetic characteristic index value includes:
acquiring contribution degree information that indicates a contribution degree, which is a magnitude of contribution of each genetic mutation to a genetic characteristic; and
computing the second score only for a genetic mutation whose contribution degree is equal to or higher than a threshold among genetic mutations other than the mutation of interest.

6. The genetic characteristic estimation device according to claim 5,
wherein the computation of the genetic characteristic index value includes computing the first score only for the mutation of interest whose contribution degree is equal to or higher than the threshold.

7. The genetic characteristic estimation device according to claim 1, wherein the genetic characteristic index value is a polygenic risk score.

8. A control method executed by a computer, the control method comprising:
acquiring genetic mutation information and position information, the genetic mutation information indicating a genetic mutation in a deoxyribonucleic acid (DNA) sequence of a target cell obtained from a target organism, the position information associating a position in the DNA sequence with a type of a cell or a type of an organ; and
determining a mutation of interest, which is a genetic mutation at a position that the position information associates with the type of the target cell or the type of the organ having the target cell, from the genetic mutations indicated by the genetic mutation information; and
computing a genetic characteristic index value based on a characteristic of the mutation of interest, the genetic characteristic index value representing a genetic characteristic of the target organism.

9. The control method according to claim 8,
wherein the position indicated by the position information represents a promoter, an enhancer, a chemically modified region, or a region of a specific gene in a DNA that the cell associated with that position has or a cell of the organ associated with that position has.

10. The control method according to claim 8,
wherein the computation of the genetic characteristic index value includes:
acquiring contribution degree information that indicates a contribution degree, which is a magnitude of contribution of each genetic mutation to the genetic characteristic; and
computing the genetic characteristic index value based on the characteristic of the mutation of interest whose contribution degree is equal to or higher than a threshold.

11. The control method according to claim 8,
wherein the computation of the genetic characteristic index value includes:
computing a first score based on the characteristic of the mutation of interest and a second score based on a characteristic of a genetic mutation other than the mutation of interest;
assigning different weights to the first score and the second score in such a way that an influence of the first score on the genetic characteristic index value is larger than an influence of the second score on the genetic characteristic index value; and
computing the genetic characteristic index value by using the first score and the second score to which the weights are assigned.

12. The control method according to claim 11,
wherein the computation of the genetic characteristic index value includes:
acquiring contribution degree information that indicates a contribution degree, which is a magnitude of contribution of each genetic mutation to the genetic characteristic; and
computing the second score only for a genetic mutation whose contribution degree is equal to or higher than a threshold among genetic mutations other than the mutation of interest.

13. The control method according to claim 12,
wherein the computation of the genetic characteristic index value includes computing the first score only for the mutation of interest whose contribution degree is equal to or higher than the threshold.

14. The control method according to claim 1, wherein the genetic characteristic index value is a polygenic risk score.

15. A non-transitory computer-readable medium storing a program for causing a computer to execute:
acquiring genetic mutation information and position information, the genetic mutation information indicating a genetic mutation in a deoxyribonucleic acid (DNA) sequence of a target cell obtained from a target organism, the position information associating a position in the DNA sequence with a type of a cell or a type of an organ; and
determining a mutation of interest, which is a genetic mutation at a position that the position information associates with the type of the target cell or the type of the organ having the target cell, from the genetic mutations indicated by the genetic mutation information; and
computing a genetic characteristic index value based on a characteristic of the mutation of interest, the genetic characteristic index value representing a genetic characteristic of the target organism.

16. The computer-readable medium according to claim 15,
wherein the position indicated by the position information represents a promoter, an enhancer, a chemically modified region, or a region of a specific gene in a DNA that the cell associated with that position has or a cell of the organ associated with that position has.

17. The computer-readable medium according to claim 15,
wherein the computation of the genetic characteristic index value includes:
acquiring contribution degree information that indicates a contribution degree, which is a magnitude of contribution of each genetic mutation to the genetic characteristic; and
computing the genetic characteristic index value based on the characteristic of the mutation of interest whose contribution degree is equal to or higher than a threshold.

18. The computer-readable medium according to claim 15,
wherein the computation of the genetic characteristic index value includes:
computing a first score based on the characteristic of the mutation of interest and a second score based on a characteristic of a genetic mutation other than the mutation of interest;
assigning different weights to the first score and the second score in such a way that an influence of the first score on the genetic characteristic index value is larger than an influence of the second score on the genetic characteristic index value; and
computing the genetic characteristic index value by using the first score and the second score to which the weights are assigned.

19. The computer-readable medium according to claim 18,
wherein the computation of the genetic characteristic index value includes:
acquiring contribution degree information that indicates a contribution degree, which is a magnitude of contribution of each genetic mutation to the genetic characteristic; and
computing the second score only for a genetic mutation whose contribution degree is equal to or higher than a threshold among genetic mutations other than the mutation of interest.

20. The computer-readable medium according to claim 19,
wherein the computation of the genetic characteristic index value includes, computing the first score only for the mutation of interest whose contribution degree is equal to or higher than the threshold.

21. (canceled)
